(19) United States
(12) Patent Application Publication
(10) Pub. No.: US 2008/0242979 A1
(43) Pub. Date: Oct. 2, 2008

(54) Combined X-ray detector and ultrasound imager

(76) Inventors: Rayette Ann Fisher, Niskayuna, NY (US); Kai Erik Thomenius, Clifton Park, NY (US); Charles Steven Korman, Niskayuna, NY (US)

Correspondence Address: GENERAL ELECTRIC COMPANY (PCPI);C/O FLETCHER YODER, P. O. BOX 692289, HOUSTON, TX, 77269-2289, US

(21) Appl. No.: 11/731,327

(22) Filed: Mar. 30, 2007

(51) Int. Cl. A61B 6/00

(52) U.S. Cl. 600/427

(57) ABSTRACT

An imaging system is disclosed that includes a first imaging panel and a second imaging panel disposed about an imaging volume. The imaging panels may be configured to image the entire imaging volume and may include panels using any form of acoustic or electromagnetic energy such as ultrasound panels, optical panels, electrical impedance panels, field emitter/x-ray detector panels, or a combination thereof. In one embodiment, a first group of sensors are included in a 2D matrix of sensors configured to transmit ultrasound through the imaging volume to a second group of sensors included in a second 2D matrix of sensors and vice versa. In a second embodiment the system may further include a second imaging system having a transmitter, a receiver, or both, disposed adjacent the first imaging panel, the second imaging panel, or both. A third embodiment may include at least one additional imaging panel.

### BACKGROUND

The present invention relates to a dual modality imaging system. More specifically, embodiments of the present invention relate to a combined ultrasound and X-ray imaging system.

In modern healthcare facilities, medical diagnostic and imaging systems are used for identifying, diagnosing, and treating diseases. Diagnostic imaging refers to any visual display of structural or functional patterns of organs or tissues for a diagnostic evaluation. One diagnostic imaging technique is ultrasound. An ultrasound imaging system uses an ultrasound sensor or transducer for transmitting ultrasound signals into an object, such as the breast of the patient being imaged, and for receiving reflected ultrasound signals back into the same transducer. The reflected ultrasound signals received by the ultrasound sensor are processed to reconstruct an image of the object. Unfortunately, existing sensors must be physically moved across the object to generate an image, and thus, measurement errors may result due to movement of the object among other things. Moreover, as a result of the movement delay time, the overall scan does not produce an instantaneous or real-time image of the region of interest of the object. These problems can complicate registration with images from other modalities.

For example, another diagnostic imaging technique is mammography, by which a breast of a patient may be non-invasively examined or screened to detect abnormalities, such as lumps, fibroids, lesions, calcifications, and so forth. Typically mammography employs specialized radiographic techniques to generate images representative of a breast tissue. A mammography imaging system typically comprises an X-ray imaging system, which uses a source of radiation, such as an X-ray source, a breast-positioning sub-system, an X-ray detector for imaging, data acquisition computers, control software and display monitors. Again, the foregoing problems with existing ultrasound systems can complicate registration between these two modalities.

### BRIEF DESCRIPTION

Certain embodiments of the present invention include an imaging system having a first imaging panel and a second imaging panel disposed about an imaging volume. The first and second imaging panels include a two-dimensional (2D) matrix of sensors configured to transmit, receive, or both, alone or in various combinations with one another. The imaging panels may be configured in various shapes with the 2D matrix of sensors configured to the shape of the imaging panel. For example, the imaging panel may be flat and rectangular or may be an arcuate, semi-circular, or circular. The 2D matrices of sensors or transducer may include at least 4,000,000 capacitive micromachined ultrasonic transducers (cMUTs), polyvinylidene flouride (PVDF) sensors, cadmium zinc telluride (CZT) sensors, field emitters, x-ray detectors, piezoelectric transducers (PZTs), piezoelectric micromachined ultrasonic transducers (pMUTs), photoacoustic detectors, optical detector arrays hydrophones, or a combination thereof. The imaging panels may be configured to image the entire imaging volume, which may include an area of at least 4 centimeters by at least 4 centimeters, and/or discrete portions without moving the sensors. The imaging panels may include ultrasound panels, photoacoustic panels, optical panels, electrical impedance panels, field emitter/x-ray detector panels, sound panels, or a combination thereof.

In one embodiment, a first group of sensors included in the first 2D matrix of sensors is configured to transmit ultrasound through the imaging volume to a second group of sensors included in the second 2D matrix of sensors and vice versa. The first and second groups of sensors may be offset from one another in a direction generally parallel to the first or second imaging panel. Moreover, the first group of sensors may be configured to transmit and receive ultrasound with respect to a portion of the imaging volume, and the second group of sensors may be configured to transmit and receive ultrasound with respect to the same portion of the imaging volume. Furthermore, the first and second group of sensors may be configured to transmit and receive ultrasound with respect to a portion of the imaging volume that is located perpendicular and/or at an oblique angle to the groups of sensors.

In a second embodiment the system may further include a second imaging system having a transmitter, a receiver, or both, disposed adjacent to the first imaging panel, the second imaging panel, or both. The second imaging system may be an X-ray system and the system may include image combining circuitry configured to reconstruct a co-registered dual modality image. Additionally, the second imaging system may be an impedance system that includes a first and second set of electrodes disposed about the imaging volume. The first set of electrodes is configured to apply an electric current and the second set of electrodes is configured to measure the resulting change in the voltage potential. In alternate configurations, these could be current sources and detectors.

A third embodiment may include at least one additional imaging panel. The additional imaging panel may also include a two-dimensional (2D) matrix of sensors configured to transmit, receive, or both, alone or in various combinations with the other imaging panels. Furthermore, the additional imaging panel may be disposed about the imaging volume and configured to image the entire imagine volume and/or discrete portions of the imaging volume without moving the sensors.

In certain embodiments, the imaging panel may include a sensor array coupled to an electronics array. The electronics array may include a plurality of integrated circuit chips stacked on top of the sensor array. The plurality of integrated chip may include an aperture control block, image processing block, power management block, transmit/receive block, beam formation block, audio block, display block, or a combination thereof. Furthermore, the first and second 2D matrices of sensors may electronically scan the volume via changing the size and shape of a group of sensors and/or by using different transmit and receive delays in coordination with each group of sensors.

In certain embodiments, the imaging panels may be configured to perform through transmission imaging which includes transmitting a signal from one panel through an imagining volume to one or more additional panels that then receive the signal. The process may be repeated in a specified or calculated manner (i.e., an image cycle) with additional panels transmitting and receiving through the imaging volume. The image cycle may follow a predetermined sequence and time interval or may be adjusted by the operator. Once the image cycle is complete, a multiple volumetric image, from current or previous studies, may be co-registered and the signals processed to remove speckle and shadowing artifacts. Embodiments of through volume transmission may include using the first and second 2D matrices of sensors to measure the time to transmit and receive between the imaging panels, as well as, to measure the attenuation of each transmit beam as it passes from one imaging panel to another.

In certain embodiments, the imaging panels may be configured to compare differences in volumetric shear and bulk strain images and B-mode images prior to and after compressing the imaging volume. Furthermore, one imaging panel may transmit a high-energy acoustic pulse to compress the tissue and one or more imaging panels may measure volumetric strain and B-mode images prior to and after the high-energy pulse. Similarly, on subsequent volumetric imaging cycles a different panel could provide the high-energy pulse. Additionally, the first and second 2D matrices of sensors may be configured to produce acoustic energy that is capable of ablating tissue, eliminating tumors, or destroying microbubbles in a tumor region to facilitate drug delivery.

Finally, in certain other embodiments, one or more of the panels may be configured to transmit light energy into the tissue while one or more panels may be configured to receive resultant acoustic energy. Alternatively, one panel may be configured to generate a beat frequency while one or more additional panels may be configured to receive acoustic echoes resultant from the beat frequency interrogation of the volume. Further, as illustrated by the discussion regarding the third imaging panel, certain embodiments may include additional imaging panels and matrices of sensors (i.e., fourth, fifth, etc.) to increase functionality. Therefore, embodiments of the present invention are by no means limited to just two imagining panels or two 2D matrices of sensors.

### DRAWINGS

These and other features, aspects, and advantages of the present invention will become better understood when the following detailed description is read with reference to the accompanying drawings in which like characters represent like parts throughout the drawings, wherein:

FIG. 1 is a perspective view of the imaging system;

FIG. 2 is block diagram of the imaging system illustrated in FIG. 1;

FIG. 3 is an elevational view of the imaging system illustrating the installation and removal of the imaging panels;

FIG. 4 is a cross-sectional view of an individual sensor or transducer;

FIG. 5 is a perspective view of the sensor illustrated in FIG. 4;

FIG. 6 is a perspective view a subelement or acoustic group that includes a plurality of the sensors illustrated in FIG. 5;

FIG. 7 is a perspective view a sensor subarray that includes a plurality of the subelements illustrated in FIG. 6;

FIG. 8 is a perspective view a sensor module that includes a plurality of the sensor subarrays illustrated in FIG. 7;

FIG. 9 is a perspective view a sensor array or subset that includes a plurality of the sensor modules illustrated in FIG. 8;

FIG. 10 is an elevational view of the sensor array shown in FIG. 9, illustrating one possible grouping of the subelements or acoustic groups;

FIG. 11 is a block diagram illustrating the sensor array, shown in FIG. 9, coupled to the electronics array and integrated circuit chips;

FIG. 12 is a perspective view of a sensor assembly that includes a plurality of the sensor arrays and electronic arrays illustrated in FIG. 9;

FIG. 13 is a perspective view an imaging panel that includes a plurality of sensor assemblies as illustrated in FIG. 12;

FIG. 14 is a perspective view illustrating the beam forming technique of beam scanning;

FIG. 15 is a perspective view illustrating the beam forming technique of beam scaling; and

FIG. 16 is a perspective view illustrating the beam forming technique of beam steering.

### DETAILED DESCRIPTION

As discussed in further detail below, various embodiments of an imaging system are provided. The imaging system may include multiple modalities and may be configured to produce a co-registered dual modality image without requiring the relocation of the imaging volume. The modalities may include an ultrasound systems, X-ray imaging systems (including mammography system), molecular imaging systems, computed tomography (CT) systems, positron emission tomography (PET) systems, magnetic resonance imaging (MRI) systems, and electric impedance imaging systems. The imaging system may be configured to electronically scan the image volume without moving the sensors via a first imaging panel and a second imaging panel disposed about the imaging volume. The imaging panels may include a 2D matrix of sensors that may be configured to transmit, receive, or both, (e.g., ultrasound) alone or in various combinations with one another. The 2D matrix may be further subdivided into groups of sensors or transducers that may be configured to transmit, receive, or both through the imaging volume to another group of sensors located in another 2D matrix of sensors. The group of sensors may be configured to electronically scan the entire image volume or portions thereof without moving the sensors. The electronic scan may be conducted via a reconfigurable array making use of a number of different beam forming techniques, such as beam translation, beam scaling, and/or beam steering.

The imaging panels may further include a sensor array and an electronics array. The sensor array may include a number of individual sensors or groups of sensors configured to interface with an electronics array. In one embodiment, the number of individual sensors may include at least 4,000,000 and the system may be configured to electronically scan an area of at least 4 centimeters by at least 4 centimeters, and/or discrete portions of the image volume without moving the sensors. In a second embodiment, the number of individual sensors may include at least 8,000,000 and the system may be configured to electronically scan an area of at least 8 centimeters by at least 4 centimeters, and/or discrete portions of the image volume without moving the sensors. Further, additional embodiments may include various numbers of individual sensors and may be configured for various applications. For example, one application might be configured for scanning bones in an ankle (i.e., a very small area requiring fewer individual sensors) where the other might be configured for scanning an entire breast (i.e., a larger area requiring a larger number of individual sensors). Therefore, certain embodiments may include fewer than 4,000,000 sensors (e.g., 1,000,000 sensors) and scan smaller areas than 4 centimeters by 4 centimeters (e.g., 2 centimeters by 2 centimeters). Additionally, the electronics array may include a number of integrated circuit chips. For example, the integrated circuit chips may include an aperture control block, image processing block, power management block, transmit/receive block, beam formation block, audio block, display block, or a combination thereof.

Finally, the 2D matrices of sensors may be configured to image and eliminate tumors in the imaging volume by electronically scanning the volume via changing the size and shape of a groups of sensors, by using different transmit and receive delays in coordination with image combinations, by comparing differences in the image prior to and after compressing the imaging volume, by measuring the acoustic transmit time between the imaging panels, by ablating tissue and/or removing lesions, or a combination thereof. These features introduced above are now discussed in further detail below with reference to the figures.

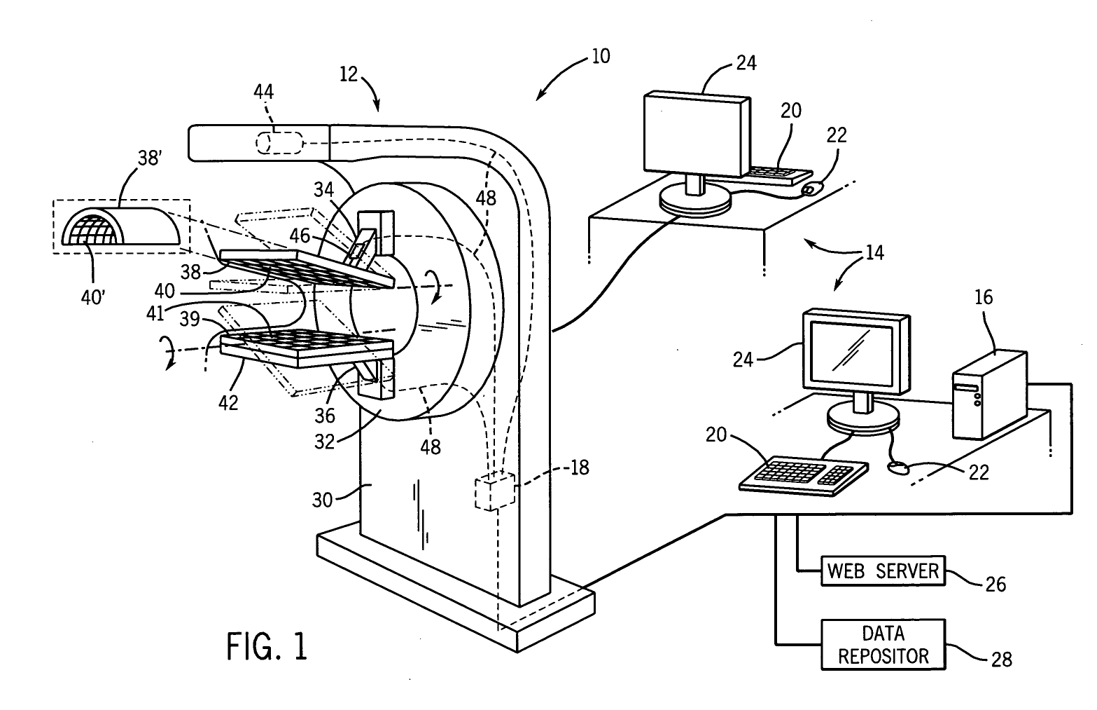

Turning now to the drawings, and referring first to FIG. 1, an exemplary imaging system 10 is illustrated that generally includes an imaging station 12 and one or more workstations 14. The workstations 14 may include a computer unit 16 independent from the imaging station 12, or a computer unit 18 integrated into the imaging station 12, and/or both. Each workstation may include user interface devices, such as a keyboard 20, a mouse 22, and/or a monitor or display 24. These devices enable a user to control the imaging station 12, receive image data from the imaging station 12, and/or process the image data. Additionally, the interface devices 20, 22 and the display 24 may also be integrated into the imaging station 12. Furthermore, the imaging station 12 and/or workstation 14 may be connected to a network. The network may include a web server 26 enabling for remote access to the imaging system 10 via the network. Further, the network may include a data repositor 28 for retrieving or storing images via either the imaging station 12 or the workstation 14.

The imaging station 12 generally includes a base 30 and a rotating gantry 32. The rotating gantry includes a top arm 34 and a bottom arm 36 positioned opposite of one another. A first imaging panel 38 is connected to the top arm 34 and a second imaging panel 39 is attached to the bottom arm 36. The imaging panels 38, 39 may be configured to jointly or independently rotate about a perpendicular and/or a parallel axis and may be movably fixed about an imaging volume or tissue volume placed there between. A first two-dimensional (2D) matrix of sensors or 40 and a second 2D matrix of sensors or 41 are located on the respective imaging panels 38, 39. The 2D matrix of sensor will be discussed in more detail below. Additionally, the imaging panels may be configured in various shapes with the 2D matrix of sensors 40, 40′ configured to the shape of the imaging panel. For example, the imaging panel may be generally flat and rectangular 38 or may be an arcuate, semi-circular, or circular shape 38′. As discussed in further detail below, additional imaging panels (not pictured) may be included that also rotate jointly and/or independent from the first two imaging panels. Each imaging panel may include ultrasound panels, optical panels, electrical impedance panels, sound panels, or a combination thereof.

The imaging system 10 may also include a second or another imaging modality that further includes a detector 42 and a source 44. For example, the detector 42 may be an X-ray detector and the source 44 maybe an X-Ray source. In one embodiment, the source 44 may be positioned in the upper portion of the base 30 and configured to transmit radiation energy through the imaging volume to the detector 42. In other embodiments, the source may be positioned orthogonal to the panels. As discussed above, the dual modality enables the user the capability of producing co-registered images without have to reposition the imaging volume 50. Moreover, the use of the imaging panels 38, 39 and 2D matrices of sensors 40, 41 enables the user to acquire this co-registered image without having to mechanically move the matrices of sensors 40, 41 to scan the entire imaging volume. In certain embodiment, the radiation energy generated by the second modality may pass through the imaging panels 38,39. Therefore, in at least one embodiment, the panels may include electronics that are designed to handle the radiation energy without significantly damaging the electronic components. As will be discussed below, in other embodiments the imaging panels may be removed during the second imaging process.

The imaging station 12 may also include connectors 46 that couple the imaging panel 38, 39 to the workstation 14 via electrical conductors 48. The connectors 46 enable the user to disconnect the imaging panel 38, 39 for service and/or repair. Additionally, the connectors 46 enable the user to remove the imaging panel 38, 39 in the situation where the operation of a second modality might affect the electronics included in the imaging panel 38, 39. For example, ultrasound imaging panels may have radiation sensitive electronics that could limit the life or functionality of such panels if subjected to the radiation energy of an X-ray source. However, as discussed above, one embodiment of the present invention is enabled with radiation hard electronics and does not require the imaging panels 38, 39 to be removed when the second modality is an X-ray imaging process or any other modality that could impact the functionality of the panels 38, 39. Additionally, the imaging panels 38, 39 may encompass a number of different embodiments containing a number of different sensors which will be discussed in more detail below. In some cases, one of these embodiments may be preferable over another for the particular imaging application. In these cases, the connectors 46 enable the user to conveniently replace the imaging panels 38, 39 or 2D matrices of sensors 40, 41 to the configuration with the preferred imaging panels and/or sensors.

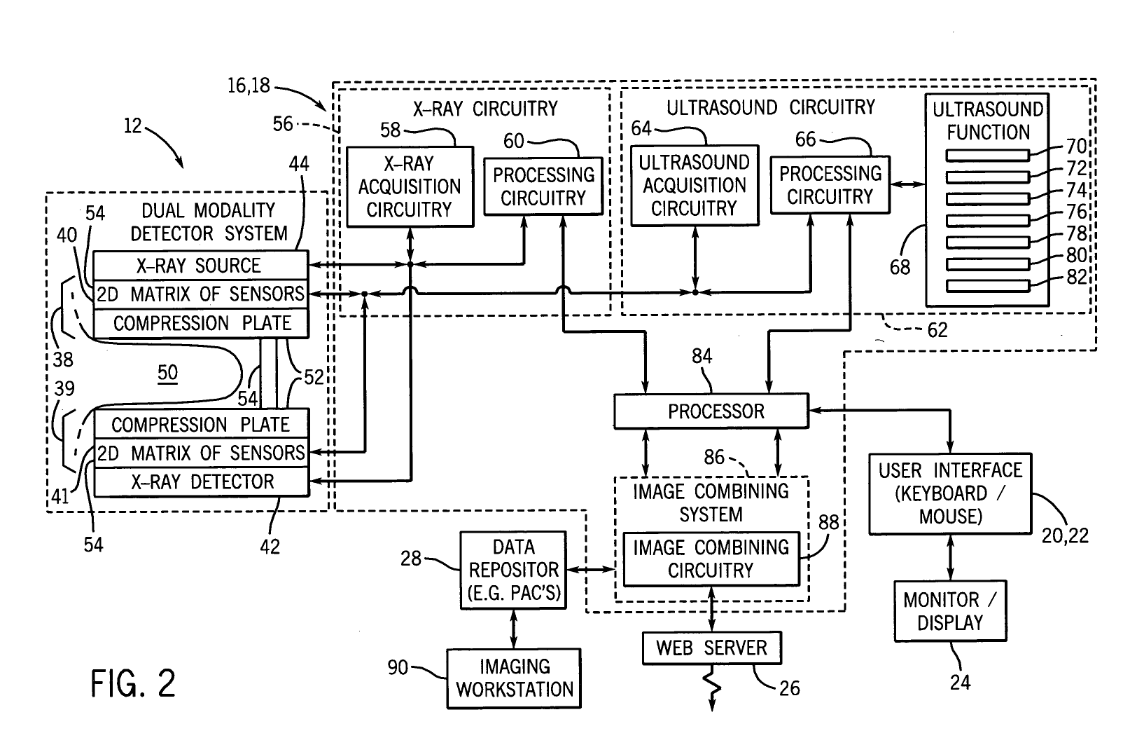

A block diagram of the system is illustrated in FIG. 2. An imaging volume or tissue volume 50 is shown positioned between two imaging panels 38, 39. Generally, the imaging panels 38, 39 include a compression plate 52 and 2D matrices of sensors 40, 41. As discussed, the imaging panels 38, 39 are not limited to a specific modality and the 2D matrices of sensors 40, 41 may be independent of the compression plate to allow for the removal of the matrix 40, 41 without having to reposition the tissue volume 50 placed between the compressions plates 52. In other words, the tissue volume 50 can remain undisturbed while the 2D matrices of sensors 40, 41 are removed from the path of the radiation. Furthermore, positioning the tissue volume 50 between the two independent matrices of sensors 40, 41 enables for the transmission of the signal through the entire tissue volume. In other words, one 2D matrix 40 can transmit the signal to the oppositely positioned matrix 41 and vice versa. This is not possible when only one 2D matrix is present which requires the single 2D matrix to provide both transmission and reception of the signal. Thus, having at least two 2D matrices of sensors 40, 41 enables for a number of increased functionalities that are discussed in more detail below.

The 2D matrices of sensors 40, 41 may be further subdivided into groups of sensors or transducers. These groups of sensors may be configured to transmit, receive, or both through the imaging volume to another group of sensors located in another 2D matrix of sensors. The group of sensors may be configured to electronically scan the entire image volume or portions thereof without moving the sensors. Additionally, the first and second groups of sensors maybe offset from one another in a direction generally parallel to the first or second imaging panel. Moreover, the first and second group of sensors may be configured to transmit and receive ultrasound with respect to a same portion of the imaging volume albeit from different orientations, e.g., (X, Y, Z) coordinates, angles, distances, and so forth. For example, the first and second group of sensors may be configured to transmit and receive ultrasound with respect to a portion of the imaging volume that is located at an oblique angle to the groups of sensors. Thus, when combined with signal transmission techniques, the through transmission enables for a 360 degree view of the image volume.

Additionally, one embodiment of the present invention includes at least one additional imaging panel 54 that includes a third 2D matrix of sensors. The additional matrix may transmit and receive signals to the other two 2D matrix of sensor, thus increasing the transmission/reception group of sensors disposed about the imaging volume. As this embodiment illustrates, the present invention is not limited to only two 2D matrices of sensors 40, 41 and may incorporate a number of 2D matrices of sensors positioned around the image volume 50.

Finally, the second modality detector 42 and source 44 are also shown positioned on opposite sides of the imaging volume 50 allowing for the transmission of the signal through the imaging volume 50. However, depending on the modality, the detector 42 and source 44 do not necessarily have to be positioned on opposite sides of the image volume 50.

As discussed, the imaging station 12 is coupled to the workstation 14 (e.g., computer units 16, 18) that includes the corresponding modality and imaging circuitry. In one embodiment, the workstation includes X-ray circuitry 56 having an X-ray acquisition system 58 and processing circuitry 60 used to operate and produce the X-ray image. This embodiment may also include ultrasound circuitry 62 having an ultrasound acquisition circuitry 64 and processing circuitry 66. The acquisition and processing circuitry may include a number of functionalities, for example, three-dimensional (3D) imaging 70, high frequency resolution 72, Doppler imaging 74, speed of sound 76, attenuation 78, elastography 80, and high intensity focused ultrasound 82, each of which is discussed in more detail below.

As discussed above, one embodiment has imaging panels 38, 39 disposed about opposite sides of the imaging volume 50. This allows for through imaging of the imaging volume 50. In other words, the ultrasound signal is transmitted from one side of the imaging volume, via the first imaging panel 38, through the volume 50 to the opposite of side of the imaging volume where it is received, via the second imaging panel 39, and vice versa. This enables the imaging system 10 to perform 3-D image functionality 70 thereby enabling the imaging system 10 to generate a 360 degree view of the image volume 50 via the through transmission configuration.

The through transmission may also use beam forming techniques, such as beam compounding, or cross beam, to further reduce shadowing and speckle. Beam compounding includes multiple scans at different angles in the same plane which are then added together to get speckle cancellation. Furthermore, the reconfigurable sensor array, as discussed in more detail below, enables for both spatial and frequency compounding in a 360 degree manner enabling an improvement in the signal-to-noise ratio. Spatial compounding is produced by scanning at multiple angles using different transmit and receive delays and by changing the grouping shape, and then adding the imaging results thereby enabling for the cancellation of noise and improving the signal-to-noise ratio. This technique also reduces and/or eliminates shadowing because of the ability to transmit and beam steer from both imaging panels that may be positioned on either side of the imaging volume 50.

A second functionality contemplated by the present invention is high frequency resolutions 72 that enables for the detection of microcalcifications, typically on the order of 0.1 mm to 1 mm in size. These size calcifications are one of the earliest indicators of possible breast cancer. Historically, X-ray has had better resolution than ultrasound for microcalcifications detection. However, one of the embodiments makes use of cMUT sensors that enables the fine pitch ultrasound array needed to see these size microcalcifications. For example, one of the contemplated embodiments includes groups of sensors on the order of 50 to 100 um that generate ultrasound signals having a frequency of 9-15 MHz or higher in order to detect these microcalcifications. Additionally, certain embodiments may be used to detect lesions that occur near the skin by generating a 20 MHz high frequency signal. Moreover, certain embodiments enable a co-registered ultrasound and X-ray image thus enabling the high frequency resolution ultrasound modality to be used in conjunction with the X-ray modality to confirm the detection of microcalcifications.

The present invention also overcomes prior issues with ultrasound attenuation as a result of increased frequency. Generally, the ultrasound transmission becomes more attenuated by the tissue as the frequency is increased. Thus, in order to see through the entire image volume, traditional transducers typically only operated in the 5 to 10 MHz frequency range. Given the image resolution is a function of the frequency, traditional transducers typically had a lower resolution in the lateral direction. Moreover, attenuation in the axial direction was even higher and the axial resolution was even further degraded.

One of the present embodiments enables the user to transmit at a higher frequency by enabling dual imaging panels 38, 39. Because the imaging panels may be positioned on opposite sides of the imaging volume 50, the ultrasound signal only need to travel half the distance given the oppositely opposed imaging panel may scan the opposite half. Additionally, the reconfigurability of the sensor groups enables the system to obtain a higher resolution in all directions.

Doppler functionality 74 enables the operator to determine if a lesion is malignant by detecting an increased number of vessels in and around a mass through the use of color Doppler. One of the embodiments of the present invention enables for improvements in Doppler sensitivity via the use of two imaging panels. Additionally, power Doppler makes the evaluation of vascularity of breast masses easier. Again the dual imaging panel system enables an improved Doppler performance by allowing for transmission and reception around the entire lesion via the placement of the imaging panels 38, 39 on opposite sides of the imaging volume 50.

The speed of sound functionality 76 and attenuation functionality 78 enable imaging of a lesion via the physical properties of the lesion that result in a higher speed of sound or less attenuation than the surrounding tissue. The image is produced by measuring the one-way transmission and reception time from one sensor matrix to the other. Again, the dual imaging panel system enables an improved sound functionality 76 and an attenuation functionality 78 performance by enabling the user to record the arrival time of the pulse from one group of sensors located on the first imaging panel 38 to a second sensor group of sensors located on the second imaging panel 39. Additionally, a raster scan may be performed with angled beams, as well as alternating the transmission and reception plate in order to create a 360 degree tomographic reconstruction. Given the number of possible groupings of sensors, certain embodiments of present invention enable for a higher resolution and greater beam agility. Finally, embodiments of the present invention enable an operator to make attenuation measurements and correct for the attenuation caused by highly echogenic lesions that may cause shadowing. Again attenuation measurements can be made in 360 degrees and provide for another mode of imaging.

The elastography functionality 80 enables the operator to determine the elastic properties of different tissue types to help identify either benign or cancerous lesions. The rate of change of displacement of the breast tissue as a function of distance from the source of compression is called a strain image or elastogram. One elastography method is to minimally compress the breast tissue 50 and compare the ultrasound image before and after the compression using cross-correlation to determine the amount of tissue displacement due to the compression. Because benign lesions tend to be softer, they will more closely match the surrounding tissue for elastic properties, whereas cancerous lesions tend to be hard and become very visible in contrast with the surrounding tissue on an elastogram image. There are a number of techniques to generate the compression of the breast tissue 50, pushing with the hand, adding a vibration source to the transducer, or first generating a high intensity ultrasound pulse to move the tissue. All of these techniques, as well as many others, may be used with embodiments of the present invention. Again the dual imaging panel system 38, 39 enables an improved elastography imaging by allowing for transmission and reception around the entire lesion by the placement of the imaging panels 38, 39 on opposite sides of the imaging volume 50.

High Intensity Focused Ultrasound (HIFU) functionality 82 enables the operator to ablate selected tissue by focusing a high intensity ultrasound signal specifically at the tissue. HIFU may be used for the removal of uterine fibroids and as well as to eliminate many other types of tumors (e.g., breast, liver, prostate). Certain embodiments of the present invention may perform both the diagnosis functionalities discussed above, as well as the HIFU functionality. Additionally, certain embodiments of the present invention may perform the HIFU functionality with a number of different sensor types (e.g., cMUT, pMUT, etc.). Moreover, as discussed, connectors 46 enable the operator to quickly swap imaging panel 38, 39, thus allowing for a HIFU specific imaging panel to be used when desired.

Additionally, embodiments of the present invention may be used for ultrasound mediated drug therapy. For example, microbubbles with therapeutic drugs or genes located inside or attached to the bubble could be released within the insonfication field. As with the HIFU functionality, this functionality enables embodiments of the present invention to be used for therapy, as well as diagnosis. Moreover, because embodiments of the present invention have an improved resolution, a reconfigurable array, and dual imaging panels, the therapy may be more precisely controlled to the lesion site.

Another functionality or application relates to scattering and propagation effects. The proposed array arrangement or sensor grouping enables the operator to conduct further analysis using angular scattering and time reversal. With the proposed arrangement, the user may transmit from an effective point source and measure the response on the opposed imaging panel in order to determine the distortion of the spherical wavefront. The user may then retransmit the time-reversed waveform enabling the user to conduct experiments with this class of techniques. This would permit in vivo measurements of the degree of scatter or other forms of acoustic wavefront distortion. Again, the dual imaging panel system 38, 39 improves the functional performance by allowing for transmission and reception around the entire lesion by the placement of the imaging panels 38, 39 on opposite sides of the imaging volume 50.

Finally, embodiments of the present invention enable for the collection of a vast amount of data via the reconfigurable array and dual imaging panels 38, 39, thus making the computer aided diagnosis much more robust. The agility of the 2D matrices of sensors 40, 41 and wide bandwidth enabled by the sensor grouping allow for timely capture of this data without significant increase in patient measurement time. This is a result of the stationary positioning of the tissue volume 50 and the ability to quickly acquire data from the different modes of operation (e.g., attenuation, speed of sound, elastography, spatial compounding).

Embodiments of the present invention further include a processor 84 for processing the inputs and outputs between the x-ray circuitry 56, ultrasound circuitry 62, and additional sources. These additional sources may include the user interface 20, 22, web server 26, and data repository 28. Additionally, the processor 84 may be coupled to an image combing system 86 that includes image combining circuitry 88. The image combining circuitry 88 may be used to reconstruct a co-registered image based on acquired data from at least two modalities, for example, an ultrasound modality and an X-ray modality. As discussed, certain embodiments of the present invention enable the system 10 to acquire a dual modality image without having to reposition the imaging volume 50, thereby providing a spatially co-registered image. Additionally, embodiments of the present invention can be part of a network that connects the system to a data repositor (e.g., PACS) or other imaging workstations 90. Thus, the image combining circuitry may perform temporal combinations for the same patient and/or for the same or different modality images.

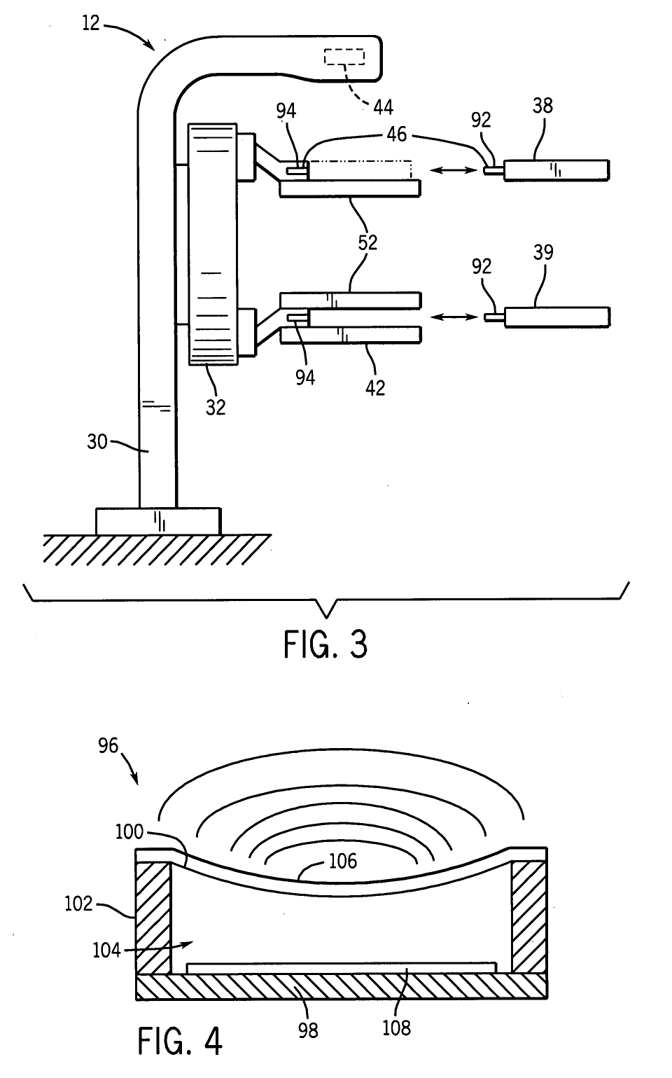

FIG. 3 is an elevational view of the imaging system 12 illustrating the installation and removal of the imaging panels 38, 39. As discussed, the imaging system 12 may have a connector 46 that enables the user to easily remove the imaging panels 38, 39 from the system 12. A first side of the connector 92 may be coupled to the imaging panel 38, 39 with the second side of the connector 94 coupled to the imaging system 12. Furthermore, the imaging panels 38, 39 may be incorporated into the compression plate 52, may be independent of the compression plate 52, or may take the place of the compression plate thereby placing the 2D matrices of sensors 40, 41 in direct contact with the imaging volume 50.

FIG. 4 illustrates a cross-sectional view of a transducer or sensor 96. A number of different sensors or transducers 96 may be used in the current system. For example, the transducer may include capacitive micromachined ultrasonic transducers (cMUTs), polyvinylidene flouride (PVDF) sensors, cadmium zinc telluride (CZT) sensors, field emitters, x-ray detectors, piezoelectric transducers (PZTs), piezoelectric micromachined ultrasonic transducers (pMUTs), photoacoustic detectors, optical detector arrays hydrophones, or a combination thereof. Two widely used types of ultrasonic transducers are cMUTs and PZTs. A PZT sensor may include a piezoelectric ceramic capable of producing electricity when subjected to mechanical stress. A cMUT transducer on the other hand, may be formed by disposing a flexible membrane disposed over a cavity in the silicone substrate. By applying an electrode to the membrane, and to the base of the cavity in the silicon substrate, and applying appropriate voltages across the electrodes, the cMUT may be energized to produce ultrasonic waves. Similarly, when appropriately biased, the membrane of the cMUT may be used to receive ultrasonic signals by capturing reflected ultrasonic energy and transforming the energy into movement of the electrically biased membrane to generate a signal.

Specifically, FIG. 4 illustrates a cross-sectional view of an exemplary cMUT transducer 96. As discussed, an array of such cMUT transducer cells may be fabricated on or within a substrate 98 and a thin membrane or diaphragm 100 is suspended above the substrate 98. The substrate 98 may be made of heavily doped silicon and the membrane 100 may be made of silicon nitride. The membrane 100 is supported by an insulating support 102, which may be made of silicon oxide or silicon nitride. A cavity 104 between the membrane 100 and the substrate 98 may be air-filled, or gas-filled, or evacuated. A layer of conductive material forms an electrode 106 on the membrane 100, and another film or layer made of conductive material forms an electrode 108 on the substrate 98. The conductive material may be aluminum alloy or other suitable conductive material. Alternatively, the bottom electrode can be formed by appropriate doping of the semiconductive substrate 98.

A capacitance is formed between the two electrodes 106 and 108 that are separated by the cavity 104. Thus, when appropriately biased, the membrane of the cMUT may be used to receive ultrasonic signals by capturing reflected ultrasonic energy and transforming the energy into movement of the membrane, thereby generating a change in the capacitance between the electrodes. The variation in the capacitance can be detected using associated electronics, thereby converting the acoustic signal into an electrical signal. Conversely, by applying appropriate voltages across the electrodes, the cMUT may be energized to produce ultrasonic waves thereby acting as a transmitter instead of a receiver.

The individual sensors 96 may have a round, rectangular, hexagonal, or other structural shapes. A cMUT cell having a square shape is shown in FIG. 5. The cMUT cells can be very small structures and typical cell dimensions are 25-50 microns measuring across one side of the cell. The dimensions of the cells are generally dictated by the designed acoustical response, and thus may be even smaller or larger than 25 microns. Additionally, FIG. 5 illustrates the conductors 110 used to couple the individual cMUT sensor to one another. The figure illustrates eight conductors 110, located on the membrane electrode 106 and extending from the center of the sensor out towards neighboring sensors (not pictured in FIG. 5). Similarly, the substrate electrode 108 has the same number of conductors enabling for the connection of the substrate electrode 108 to neighboring sensors.

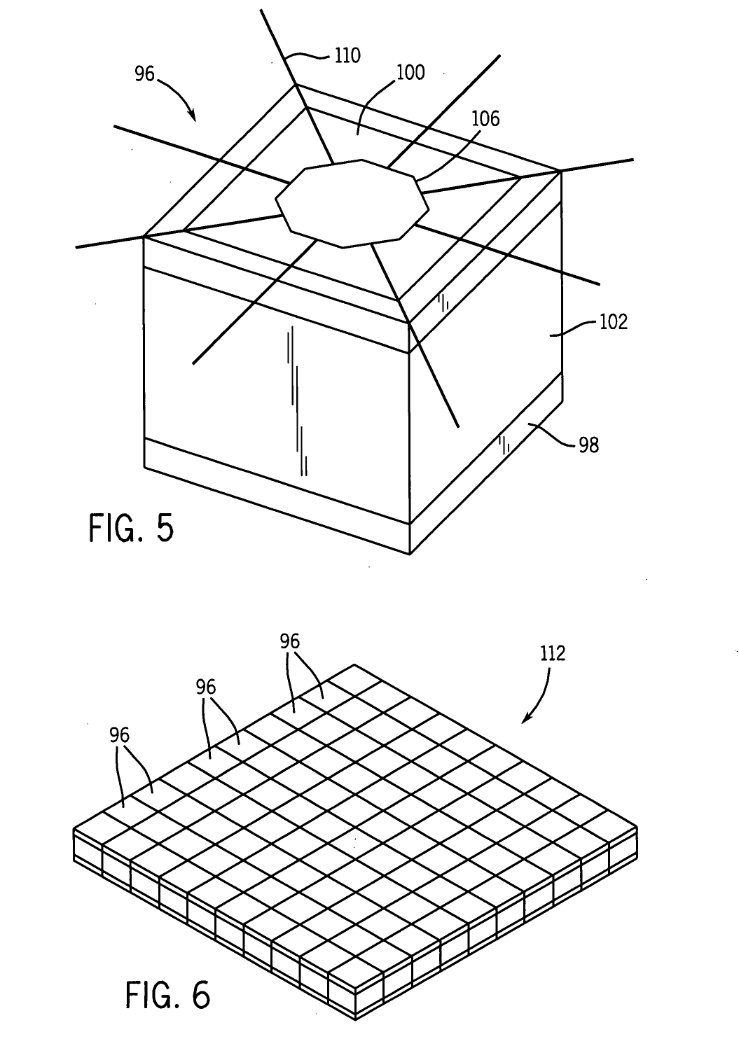

Because of the small size of the cMUT sensors they may be grouped into subelements or acoustic groups 112 and controlled as one sensor group. FIG. 6 illustrates a subelement 112 grouping showing a 10×10 sensor matrix that includes 100 individual sensors 96. Given an exemplary dimension of a 20 micron×20 micron square sensor 96, the sensor group would have a 200 micron width×200 micron height. The quality or resolution of the image formed is partly a function of the pitch of sensor groups that respectively constitute the transmission and reception apertures of the transducer array. Accordingly, to achieve high image quality, a fine pitch array of transducers is desirable for both two-dimensional and three-dimensional imaging applications. Generally, the resolution of the sensor matrix would be controlled by the subelement 112 dimension because it is the smallest individually controlled sensor group. Thus, a reduction in the sensor size or matrix size may allow for a higher resolution. Therefore, embodiments of the present invention are not limited to the 200 micron by 200 micron size resolution and may include a smaller grouping and/or smaller sensors 96.

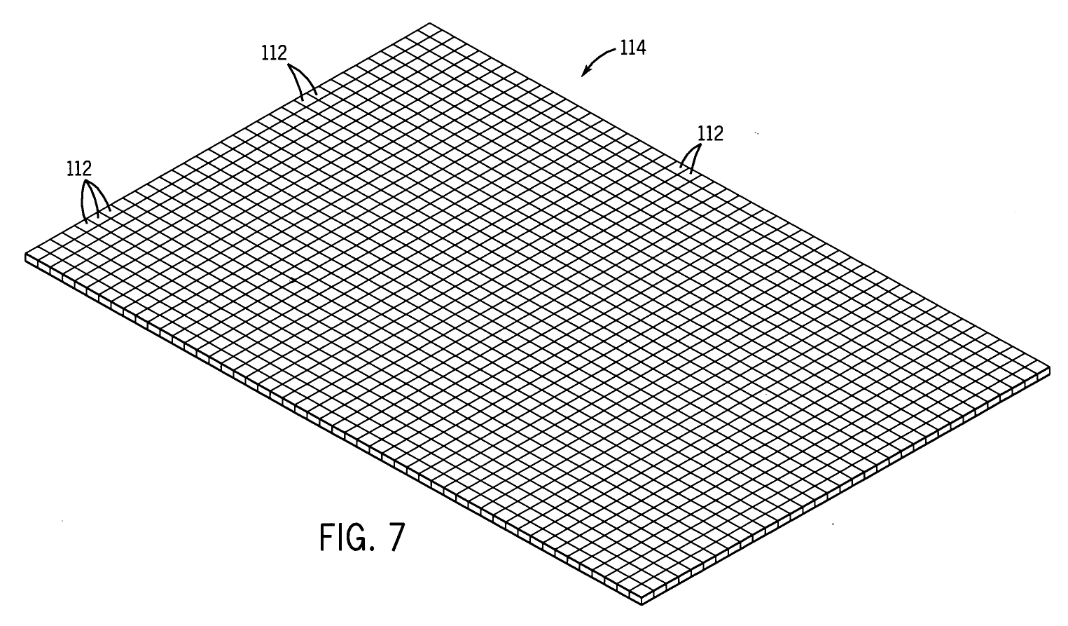

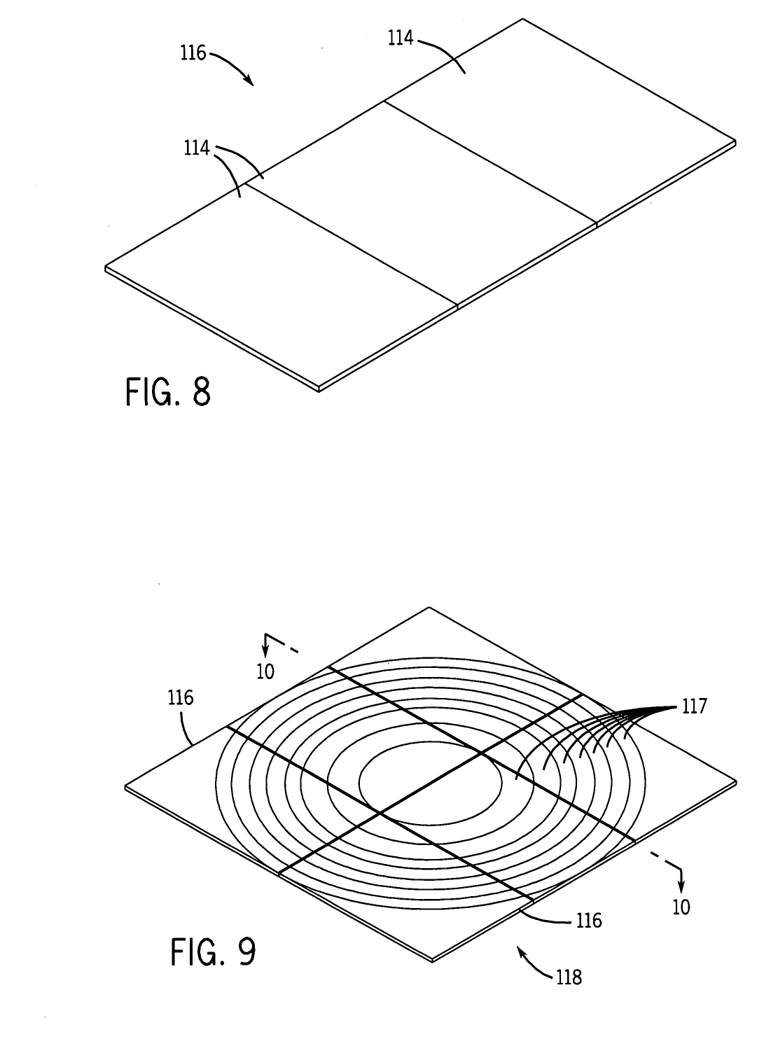

FIG. 7 illustrates a sensor subarray 114. The sensor subarray 114 is a grouping of a plurality of sensor subelements 112. The figure illustrates a 50×33 matrix which includes 1650 subelements 112, each of which includes 100 individual sensors 96, giving a total of 165,000 individual sensors in each sensor subarray 114. FIG. 8 illustrates a sensor module 116 which is a 1×3 matrix combination of sensor subarray 114. Two sensor modules 116 are then combined to form a group of sensors, group of transducers, or sensor array 118 as is illustrated in FIG. 9. Each sensor array 118 contains 990,000 individual sensors or 9,900 subelements 112. Because the sensor subelements are individually addressable, almost any type of wavefront can be created and the beam parameters can be changed on the fly. Additionally, a sensor subelement may include a single cMUT sensor.

The 2D matrices of sensors include thousands of individual sensor subelements that may be grouped as subelement rings 117 which may be disposed concentrically about one another. Additionally, the subelements may be grouped in other shapes (e.g., rectangular) and thus, the grouping is not limited to rings or circular shapes. Each group of subelement rings 117 may be configured to have the same delay and these groups can be driven by the same channel. By grouping sensor subelements, the system is not required to have a 1:1 mapping of subelements to system channels which might be size and cost prohibitive. There are a number of techniques that may be used for grouping the sensors, all of which may be used by embodiments of the present invention. One particular technique is referred to as a reconfigurable array and will be discussed in more detail below.

One sensor grouping technique contemplated by certain embodiments of the present invention is a reconfigurable array and/or mosaic array. Normally, a linear array generates an image of one plane. However, a 3D image may be generated by using the reconfigurable array to create multiple groupings of the acoustic groups or subelements 112, and then changing these groupings before and possibly during each transmit and receive event.

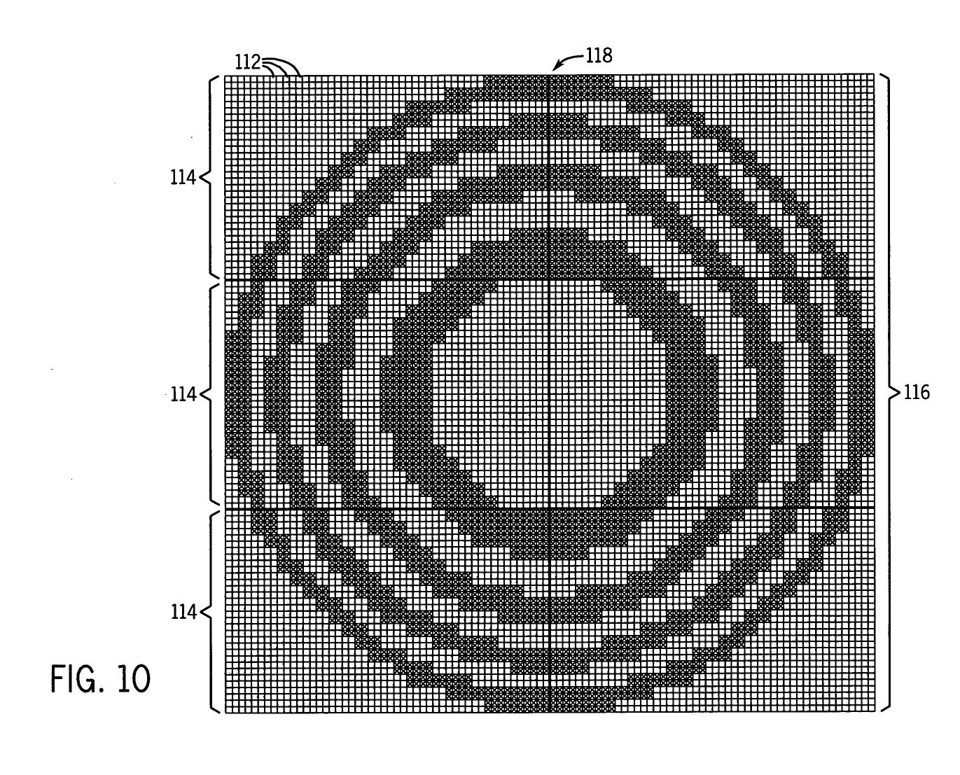

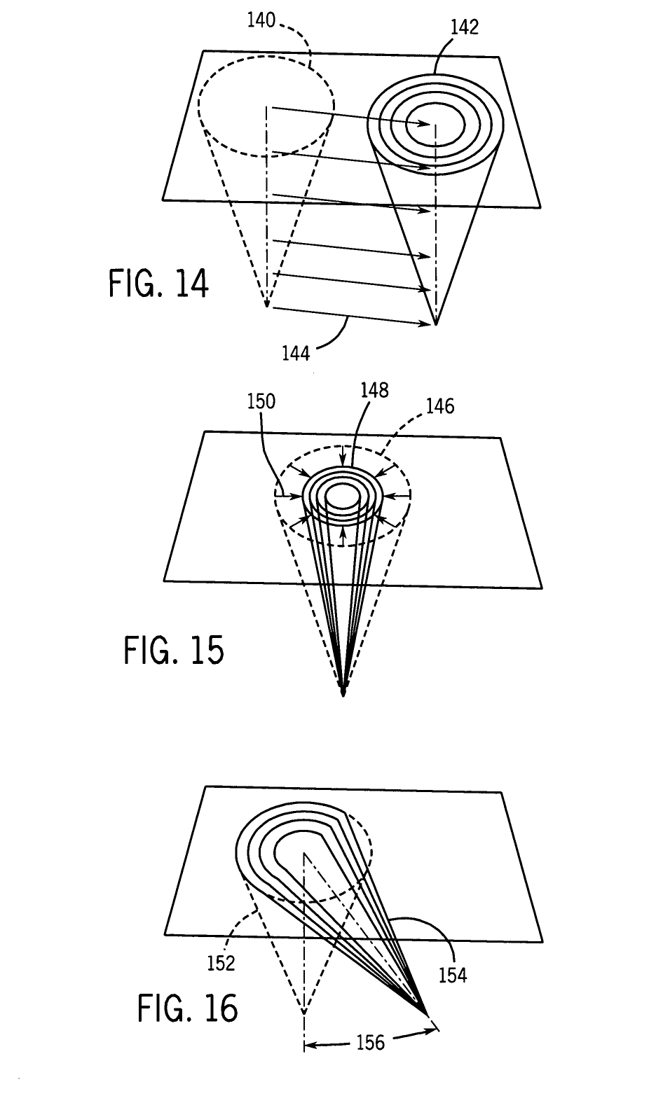

The reconfigurable array is possible because switches are located directly behind each subelement 112. This enables the system 10 to change the size and shape of each subelement ring 117 and therefore the aperture via controlling the grouping of subelements 112. The annular type of aperture, see FIG. 10, is one grouping type where the delays for each ring match the acoustic wavefront propagation. The annular aperture creates an axis-symmetric beam that enables high resolution in all directions with fewer channels. Thus, the system can perform real-time volumetric imaging over the compression plate with high resolution by electronically scanning the annular aperture across the imaging panel (see FIG. 14). The annular aperture diameters may be on the order of 20-30 cm. Additionally, the system can look beyond the plate dimensions using beam steering, as discussed in more detail below.

One of the present embodiments contemplates an imaging panel 38, 39 having dimensions of 4 centimeters by 4 centimeters. Another embodiment contemplates an imaging panel 38, 39 having dimensions of 20 centimeters by 20 centimeters. Thus, a real-time volumetric 3D image can be generated by electronically scanning the projected volume contained between the imaging panels without having to mechanically move the 2D sensor matrix 40, 41 or the imaging volume 50. It must be noted that embodiments of the present invention are not limited to these contemplated imaging panel dimensions and may incorporate larger or smaller imaging panels 38, 39.

It must be noted that all of these combinations are just one embodiment of the present invention and different sensor groupings may be used to form a sensor array 118. In one embodiment, the sensor groups or apertures are formed by connecting subelements 112 together using a switching network. The subelements 112 may be reconfigured by changing the state of the switching network. A reconfigurable ultrasound array is one that allows groups of subelements 112 to be connected together dynamically so that the shape of the resulting element can be made to match the shape of the wave front. This can lead to improved performance and/or reduced channel count.

As discussed, one form of reconfigurability is the mosaic annular array shown in FIG. 10. The mosaic annular array concept involves building annular elements by grouping subelements 112 together using a reconfigurable electronic switching network. The goal is to reduce the number of beam forming channels, while maintaining image quality and improving slice thickness. To reduce system channels, the mosaic annular array realizes that for an unsteered beam, the delay contours on the surface of the underlying two-dimensional transducer array are circular. In other words, the iso-delay curves are annuli about the center of the beam. The circular symmetry of the delays leads to the grouping of those subelements 112 with common delays and thus the annular array. The reconfigurability can be used to step the beam along the larger underlying two-dimensional sensor matrix in order to form a scan or image.

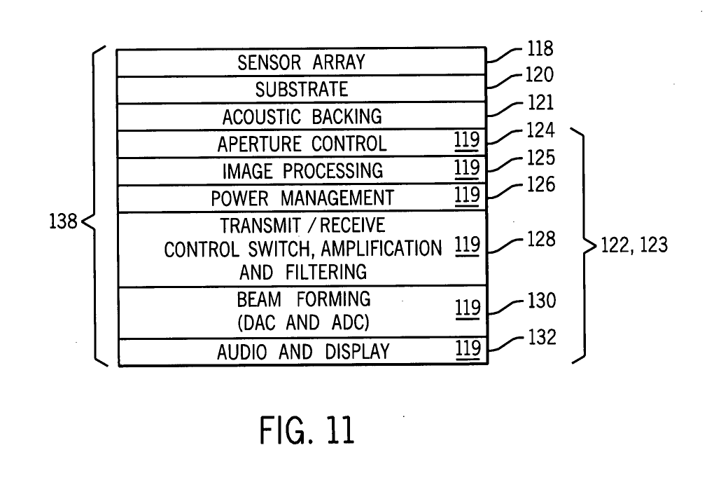

In accordance with one embodiment shown in FIG. 11, a sensor array 118 is built on a substrate 120. Suitable complementary metal oxide semiconductor (CMOS) switches and circuits are formed to produce an electronics array 122. The electronics array 122 includes a plurality of integrated circuit modules 123 that include a number of integrated circuits chips 119. The integrated circuit chips 119 are programmed for different functions that may include aperture control 124, image processing 125, power management 126, transmit/receive 128, and beam forming 130. Additionally, the electronics array 122 may include memory for storing and processing data, routines, or programs. For example, the memory may include aperture state storage to enable the system to quickly change between the transmitting state and the receiving state and vice versa.

Aperture control 124 includes switches that enable reconfiguration of the subelements 112 and the subelements connections to particular system channels, thereby enabling an aperture to be translated over the two-dimensional active area of the matrices of sensors 40, 41. The shape of the apertures is determined by such parameters as the desired focal depth, the desired steering angle for the ultrasound beam, the subelement pitch, the frequency of operation, and the number and size of the switches. Image processing 125 includes initial conditioning and filtering of the signal data. In general, most of the electronics in the array 122 include command and control of the array element and the more advanced interpretation and control is conducted by the high level processor (e.g. 60, 66). Power management 126 includes electronics configured to ensure proper biasing set-points of each element. This may include local power supply and energy storage, (e.g., microcapacitors, linear regulators) to ensure precise voltage is provided to the other support electronics. Transmit/receive 128 includes control switching, amplification, and filtering. Beam forming 130 includes digital-to-analog and analog-to digital controls. Additionally, the circuit chips may include an audio and/or video display 132. Having this type of configuration places the functionality in the imaging panels 38, 39 and reduces the number of electrical conductors 48 required to pass through the electrical connector 46. Thus, the cable harness back to the workstation 14 may be much smaller and more flexible because of the reduced number of signals (cables) needed. Finally, one or more layers of heat distribution and/or a cooling layer may be included in order to dissipate the heat away from the panels. These layers may include a finned heat sink or more complex active cooling devices. For example, a cooling fluid may be pumped through a heat sink that is located on the panel.

An acoustic backing layer 121 is preferably sandwiched between the substrate 120 and the electronics array 122 in order to improve acoustic performance of an ultrasound sensor array. In this embodiment, a vertical interconnect through the backing may be required for passage of electrical connections between the sensor array and the electronics on the next substrate. In an alternative embodiment, the sensor array may be electrically connected directly to the substrate with electronics using solder bumps, plated bumps, and interconnect interposer, anisotropically conductive epoxy. This sensor to electronics chip stack may require a next layer to be the acoustic backing. The acoustic backing material 121 may have a composition that is acoustically matched to the substrate 98, to generally block or prevent reflection of the acoustic energy back into the device. For example, if the substrate is silicon, the acoustic impedance may be approximately 19.8 MRayls.+/−0.5%.

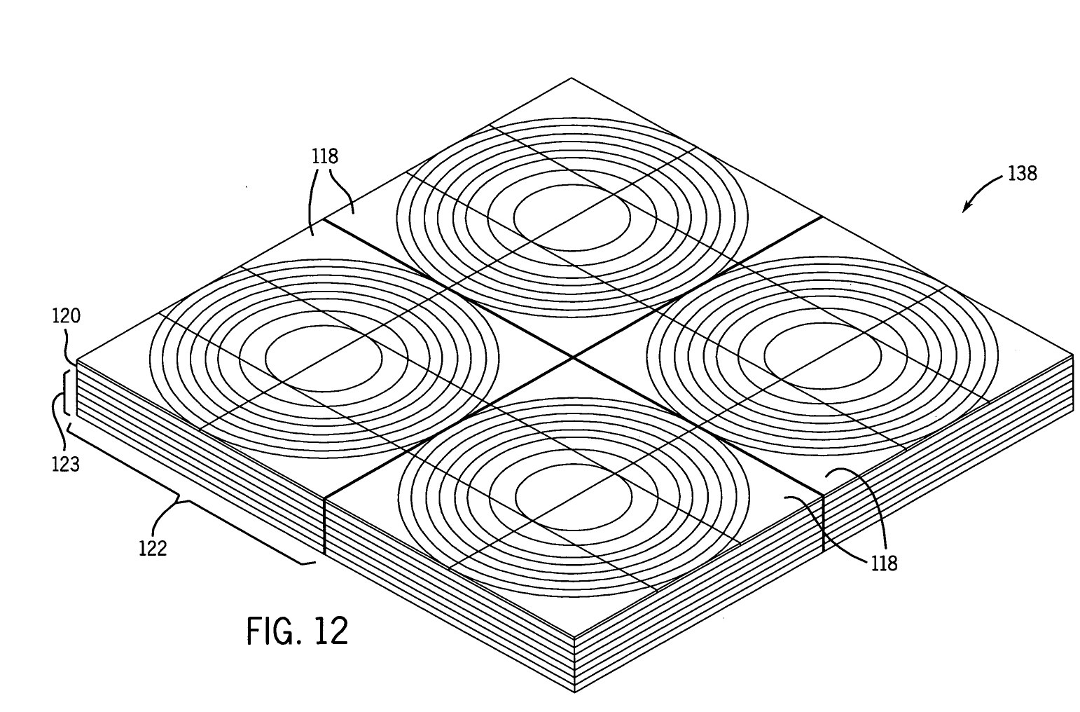

FIG. 12 illustrates a sensor assembly 138 that includes four sensor arrays 118 and four electronic arrays 122 coupled together. The illustrated sensor assembly includes 3,960,000 individual sensors 96 or 39,600 subelements or sensor groups 112. The dimension for the illustrated sensor assembly is 4 centimeters wide×4 centimeter long, but is not limited to these dimensions.

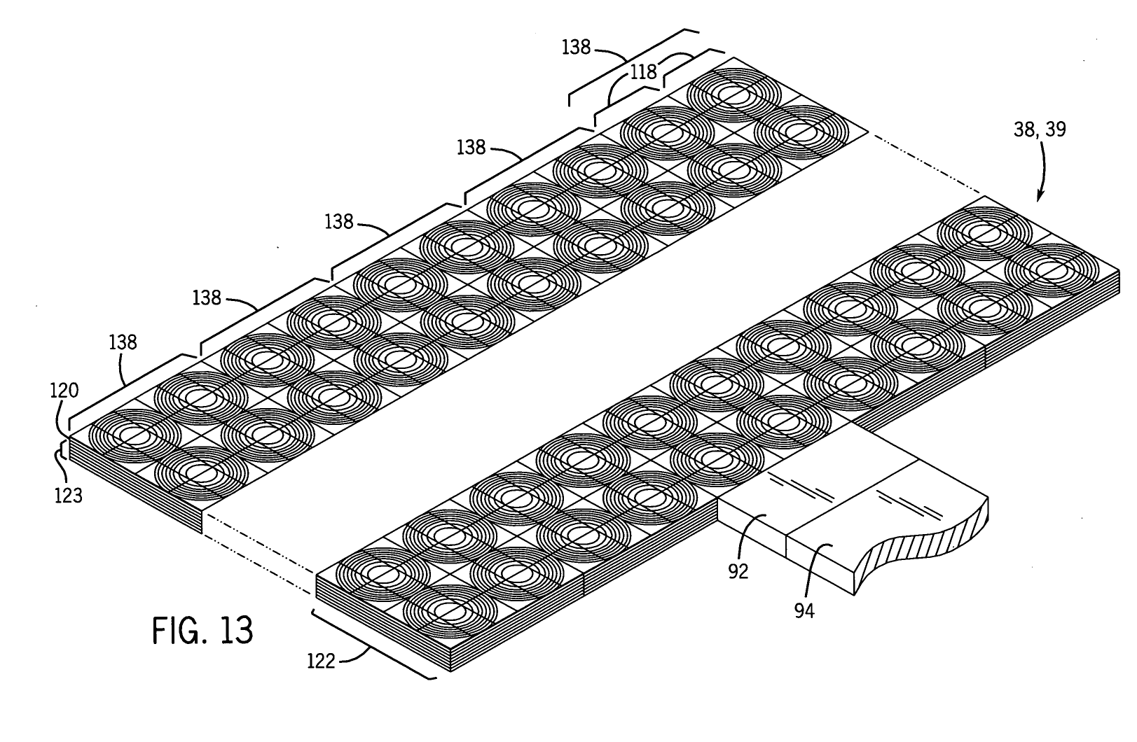

The sensor assembly 138 may be combined to form an imaging panel 38, 39 as is illustrated in FIG. 13. The imaging panel 38, 39 may be a combination of any number of sensor assemblies 138. The imaging panel 38, 39 and sensor assemblies 138 make up the 2D matrices of sensors or transducers 40, 41 that is reconfigurable into a number of groups of sensors, sensor arrays, or groups of transducers 118. One of the contemplated embodiments includes an imaging panel having a size of 20 centimeters wide by 20 centimeters long made up of a 5×5 matrix of sensor assembly 138 or a 10×10 matrix of sensor array 118. The embodiment would include 99,000,000 individual sensors 96 or 990,000 subelements or sensor groups 112. A second embodiment includes a 1×2 matrix of sensor assembly 138 or a 2×4 matrix of sensor array 118 having a size of 4 centimeters wide by 8 centimeters long. The embodiment would include 7,920,000 individual sensors 96 or 79,200 subelements or sensor groups 112.

As discussed, certain embodiments of the present invention enable the system to electronically scan the imaging volume via the reconfigurable beam and beam forming techniques. FIGS. 14-16 illustrate some of these beam forming techniques. FIG. 14 illustrates beam scanning or beam translation and includes a uniform translation of the beam forming coefficients to produce a new beam at a different location. The figure illustrates the original location of the beam, represented by the dashed lines 140, as well as the translated or new location of the beam, represented by the solid lines 142. For this example, the beam is translated to a new location as indicated by arrows 144. Repeated frequently, this generates a rectilinear two-dimensional image of the volume located above the panel. The stepping of the aperture across the imaging panel may be a gross value initially (500 um) in order to more quickly find regions of interest (ROI). Once the ROI have been detected, the system may then go back and conduct finer steps in the ROI to get more information and perform other modes of operation, as discussed above. Moreover, because the dual imaging panels 38, 39 may have a 20 centimeter×20 centimeter surface area, beam scanning enables a raster scan of the entire area. Thus, when combined with other beam forming techniques, beam scanning enables the system to generate a 3D image of the volume located between the imaging panels.

FIG. 15 illustrates beam scaling that is accomplished by changing the number of annuli and weighting of the beam coefficients. The figure illustrates the original scale of the beam, represented by the dashed lines 146, as well as the new scale of the beam, represented by the solid lines 148. For this example, the beam is scaled down to a smaller annuli diameter, as indicated by arrows 150. This enables the operator to optimize resolution at a given depth or depth range by obtaining the best f-number for the transmit aperture at that given depth or depth range. In ultrasound technology, the lateral resolution can be approximated by the product of the wavelength and the f-number. The f-number equals the depth of the focal point divided by the aperture width. Therefore lateral resolution will be best (smallest) if there is a large aperture and short wavelength (higher frequency). The trade-off occurs for resolution versus depth of penetration because the higher the frequency of the ultrasound waves the more they are attenuated in the tissue.

FIG. 16 illustrates beam steering that is accomplished by incorporating an additional bilinear term in the beam coefficients. Beam steering allows the system to direct the beam away at oblique angles (e.g., 5, 10, 15, 20, 25, 30, 35, 40, 45, 50, 55, 60, 65, 70, 75, 80, or 85 degrees) relative to a normal (e.g., 90 degrees) transmission between the panels 38, 39. The figure illustrates an unsteered beam, represented by the dashed lines 152, as well as a steered beam, represented by the solid lines 154. For this example, the beam is steered at an oblique angle that is relative to the normal of the unsteered beam, as indicated by numeral 156. This is particularly useful for generating 3-D images. Additionally, beam steering can be used at the edges to interrogate tissue that is not covered by the imaging panel 38, 39, for example, at the chest wall or where the imaging volume 50 pulls away from the imaging panels 38, 39 at the edges and/or does not have the direct contact with the imaging panels 38, 39. As discussed, a unique feature of the reconfigurable array is that a steering angle is not restricted to in-plane but can cover the entire cone-shaped 3D space in front of the plate. Moreover, beam steering may be used in Doppler imaging 74 to monitor blood flow when looking for vascularization around a lesion.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An imaging system, comprising:
a first imaging panel comprising a first two-dimensional (2D) matrix of sensors configured to transmit, receive, or both, either acoustic or electromagnetic energy alone or in various combinations with one another; and
a second imaging panel comprising a second two-dimensional (2D) matrix of sensors configured to transmit, receive, or both, either acoustic or electromagnetic energy alone or in various combinations with one another, wherein the first and second imaging panels are disposed about an imaging volume, and the first and second 2D matrices of sensors are configured to image the entire imagine volume and discrete portions of the imaging volume without moving the sensors.

2. The imaging system of claim 1, wherein the first and second imaging panels comprise a pair of opposite ultrasound panels.

3. The imaging system of claim 1, wherein the first and second imaging panels comprise a pair of opposite ultrasound panels, optical panels, electrical impedance panels, x-ray panels, sound panels, or a combination thereof.

4. The imaging system of claim 1, wherein the first and second 2D matrices of sensors comprise a plurality of capacitive micromachined ultrasonic transducers (cMUTs), polyvinylidene flouride (PVDF) sensors, cadmium zinc telluride (CZT) sensors, field emitters, x-ray detectors, piezoelectric transducers (PZTs), piezoelectric micromachined ultrasonic transducers (pMUTs), photoacoustic detectors, optical detector arrays hydrophones, or a combination thereof.

5. The imaging system of claim 1, wherein a first group of sensors in the first 2D matrix of sensors is configured to transmit ultrasound through the imaging volume to a second group of sensors in the second 2D matrix of sensors and vice versa.

6. The imaging system of claim 5, wherein the first and second groups of sensors are offset from one another in a direction generally parallel to the first or second imaging panel.

7. The imaging system of claim 5, wherein the first group of sensors is configured to transmit and receive ultrasound with respect to a portion of the imaging volume, and the second group of sensors is configured to transmit and receive ultrasound with respect to the same portion of the imaging volume.

8. The imaging system of claim 5, wherein the first and second groups of sensors are configured to transmit and receive ultrasound with respect to a portion of the imaging volume that is located at an oblique angle to the groups of sensors.

9. The imaging system of claim 1, comprising a second imaging system comprising a transmitter, a receiver, or both, disposed adjacent the first imaging panel, the second imaging panel, or both.

10. The imaging system of claim 9, wherein the second imaging system comprises an x-ray system.

11. The imaging system of claim 10, comprising image combining circuitry configured to reconstruct a co-registered dual modality image.

12. The imaging system of claim 1, wherein the first imaging panel and the second imaging panel comprise a sensor array coupled to an electronics array comprising a plurality of integrated circuit chips, and the sensor array is stacked on top of the electronics array.

13. The imaging system of claim 12, wherein the plurality of integrated circuit chips comprise an aperture control block, an image processing block, a power management block, a transmit/receive block, a beam formation block, an audio block, a display block, or a combination thereof.

14. The imaging system of claim 1, wherein the first 2D matrix of sensors comprises at least 1,000,000 sensors, and the second 2D matrix of sensors comprises at least 1,000,000 sensors.

15. The imaging system of claim 1, wherein the 2D matrices of sensors are configured to transmit, receive, or both an area of at least 4 centimeters by at least 4 centimeters without moving the sensors.

16. The imaging system of claim 1, wherein the first and second 2D matrices of sensors are configured to image the volume by electronically scanning the volume via changing the size and shape of a group of sensors, by using different transmit and receive delays in coordination with image combinations, by comparing differences in the image prior to and after compressing the imaging volume, by measuring the time to transmit and receive between the imaging panels, or a combination thereof.

17. The imaging system of claim 1, wherein the first and second 2D matrices of sensors are configured to generate acoustic energy that ablates tissue, eliminates tumors, or destroys microbubbles in a tumor region.

18. The imaging system of claim 1, comprising acquisition circuitry and processing circuitry coupled to the first and second imaging panel.

19. The imaging system of claim 1, comprising at least one additional imaging panel, wherein the at least one additional imaging panel comprises a two-dimensional (2D) matrix of sensors configured to transmit, receive, or both, alone or in various combinations with one another, and the at least one additional imaging panel is disposed about the imaging volume and configured to image the entire imagine volume and/or discrete portions of the imaging volume without moving the sensors.

20. An imaging system, comprising:
a first imaging panel comprising a first two-dimensional (2D) matrix of transducers disposed on a first side of an imaging volume; and
a second imaging panel comprising a second two-dimensional (2D) matrix of transducers disposed on a second side of the imaging volume, wherein the first and second 2D matrices of transducers are configured to probe the imaging volume without moving relative to one another.

21. The imaging system of claim 20, comprising another imaging modality comprising a source and a detector disposed on the same or opposite sides of the imaging volume.

22. The imaging system of claim 21, wherein the source comprises an x-ray source and the detector comprises an x-ray detector panel disposed adjacent the first or second imaging panel.

23. The imaging system of claim 21, wherein the source comprises a light source and the detector comprises a photoacoustic detector disposed about the imaging volume.

24. The imaging system of claim 21, wherein the source comprises a first set of electrodes configured to apply an electric current into the imaging volume and the detector comprises a second set of electrodes configured to measure the resulting change in the voltage potential, wherein the first and second set of electrodes are disposed about the imaging volume.

25. The imaging system of claim 21, wherein the source comprises an apparatus for deforming the imaging volume and the detector comprises the first 2D matrix of transducers, the second 2D matrix of transducers, or a third two-dimensional (2D) matrix of transducers.

26. The imaging system of claim 21, wherein the another imaging modality is configured to cooperate with the first and second imaging panels to generate a dual-modality image of the imaging volume.

27. The imaging system of claim 20, wherein the first and second 2D matrices of transducers a plurality of capacitive micromachined ultrasonic transducers (cMUTs), polyvinylidene flouride (PVDF) sensors, cadmium zinc telluride (CZT) sensors, field emitters, x-ray detectors, piezoelectric transducers (PZTs), piezoelectric micromachined ultrasonic transducers (pMUTs), photoacoustic detectors, optical detector arrays hydrophones, or a combination thereof.

28. The imaging system of claim 20, wherein a first group of transducers contained within the first 2D matrix of transducers is offset from a second group of transducers contained within the second 2D matrix of transducers in a direction generally parallel to the first or second imaging panel, and the first and second group of transducers are configured to transmit and receive ultrasound through a portion of the imaging volume, wherein the portion of the imaging volume is located perpendicular and/or at an oblique angle relative to at least one of the groups of transducers.

29. The imaging system of claim 20, wherein the first and second 2D matrices of transducers are configured to image and eliminate tumors in the volume by electronically scanning the volume via changing the size and shape of a groups of transducers, by using different transmit and receive delays in coordination with image combinations, by comparing differences in the image prior to and after compressing the imaging volume, by measuring the time to transmit and receive between the imaging panels, by ablating tissue and/or removing lesions, or a combination thereof.

30. A method for imaging a volume, comprising:
positioning an imaging volume between a pair of opposite imaging panels each comprising a two-dimensional (2D) set of sensors;
probing the imaging volume to obtain image data using the 2D set of sensors via transmitting, receiving, or both, alone or in various combinations, without moving the 2D set of sensors; and
generating an image of the entire imagining volume and/or a discrete portion of the imaging volume based on the image data.

31. The method of claim 30, wherein the 2-D sets of sensors comprise ultrasound panels, optical panels, electrical impedance panels, sound panels, or a combination thereof.

32. The method of claim 30, wherein probing comprises electronically scanning the volume via varying a subset grouping of the sensors, varying the transmit and receive delays to generate multiple image data that may be combined to reduce noise, comparing the difference in image data prior to and after deforming the imaging volume, measuring the time from one image panel to the other, ablating tissue and/or removing lesions, or a combination thereof.

33. The method of claim 30, comprising probing the imaging volume using a second imaging system, wherein the second imaging system comprises an x-ray system.

34. The method of claim 33, wherein generating the image comprises reconstructing a co-registered dual modality image.

35. The method of claim 30, wherein probing the imaging volume comprises generating acoustic energy that ablates tissue, eliminates tumors, or destroys microbubbles in a tumor region, the acoustic energy being generated by the 2D set of sensors or a third 2D matrices of sensors.
